(19) United States
(12) Patent Application Publication
(10) Pub. No.: US 2008/0262406 A1
(43) Pub. Date: Oct. 23, 2008

(54) SECUREMENT DEVICE FOR SHUNT CATHETER AND IMPLANTATION METHOD THEREFOR

(76) Inventor: Edward Lee Wiener, Golden Beach, FL (US)

Correspondence Address: PAUL D. BIANCO;Fleit Gibbons Gutman Bongini & Bianco PL, 21355 EAST DIXIE HIGHWAY, SUITE 115, MIAMI, FL, 33180, US

(21) Appl. No.: 12/107,932

(22) Filed: Apr. 23, 2008

Related U.S. Application Data
(60) Provisional application No. 60/913,460, filed on Apr. 23, 2007.
(60) Provisional application No. 61/027,726, filed on Feb. 11, 2008.

(51) Int. Cl. A61M 5/00

(52) U.S. Cl. 604/8

(57) ABSTRACT

The invention relates to implantable systems for securing shunt catheters. The implantable system and device functions to maintain shunt patency and thus, shunt catheter malfunction due to obstruction is prevented

### CROSS REFERENCE TO RELATED APPLICATIONS

This application claims the benefit under 35 U.S.C.119(e) of U.S. provisional patent application Ser. No. 60/913,460, filed Apr. 23, 2007, and U.S. provisional patent application, Ser. No. 61/027,726, filed Feb. 11, 2008, the contents of which are each herein incorporated by reference.

### FIELD OF THE INVENTION

The present invention relates generally to shunt catheters, particularly to cerebrospinal fluid (CSF) shunt catheters used in the treatment of hydrocephalus, and most particularly to the prevention of cerebrospinal fluid shunt catheter malfunction.

### BACKGROUND OF THE INVENTION

Hydrocephalus is the pathologic accumulation of cerebrospinal fluid (CSF) in the brain. The disease entity has a variety of clinical manifestations ranging from the more benign triad of normal pressure hydrocephlalus (gait ataxia, dementia and urinary incontinence) to those secondary to elevated intracranial pressure. The latter may follow a more malignant course and in fact, prove fatal. Hydrocephalus is treated by diverting excess cerebrospinal fluid from the brain to an alternate body cavity. Most commonly, this is the peritoneal cavity (abdominal compartment/cavity). The diversion of fluid is accomplished by a shunting device (described in the prior art, for example, in U.S. Pat. No. 4,382,445, issued May 10, 1983, to Michael Sommers and U.S. Pat. No. 5,405,316, issued Apr. 11, 1995, to Gary Magram) which is surgically implanted into the patient. The proximal limb of the shunt catheter system is introduced into the fluid cavities of the brain (ventricles) and the distal end into the peritoneal cavity (abdominal cavity). There exists an interposed valve which broadly serves to regulate CSF flow.

Cerebrospinal fluid shunts, commonly molded from silicone tubing, are susceptible to failure. In fact, 30-40% of cerebrospinal fluid shunts malfunction after primary placement in the adult population. Such malfunctions, while often benign, may prove dangerous or, if left untreated, fatal. Therefore, the maintenance of cerebrospinal fluid shunt patency is essential to patient wellness.

Presently, patients with a malfunctioning shunt undergo revision surgery to reestablish flow through the occluded shunt system. In the adult population, this often requires removal of the shunt catheter from the abdominal compartment, resecting the distal 3-4 cm of the catheter and relocating the catheter within the abdomen. The procedure and the inpatient hospitalization have associated risks and morbidity.

The majority of shunt malfunctions in the adult population are the direct result of an occlusion at the distal shunt catheter orifice (outlet). The distal catheter tip, often located in the peritoneal cavity, is susceptible to blockage by omental fat or proteinaceous debris. Also, the distal catheter may be lodged between bowel loops or between a segment of bowel and the walls of the abdominal cavity. The distal catheter may lie within an intra-abdominal fluid pocket as well. Any potential increase in outflow resistance may result in slowed CSF flow or florid obstruction of the shunt system.

In patients with normal pressure hydrocephalus, intracranial pressure ranges from 0-20 cm H2O. The mean pressure however is 8-10 cm H2O. Intracranial pressure drives cerebrospinal fluid flow through the shunt system. Additively, the opening pressure of the interposed valve, the inherent resistance of the shunt tubing and the intra-abdominal pressure equals the total outflow resistance (TOR). Intracranial pressure must overcome the TOR in order to drive cerebrospinal fluid from the brain, via the shunt system, into the abdominal compartment.

As stated previously, the majority of shunt malfunctions in the adult population occur in the abdominal compartment. While shunt valves may malfunction, this is relatively rare, occurring most often in the pediatric population. The inherent resistance of the shunt catheter is static and therefore a less likely etiology of slowed CSF flow or an acquired shunt malfunction. Unless there is an accumulation of debris within the catheter lumen (in an extra-abdominal location), the distal catheter itself is an unlikely culprit of shunt malfunction. Nevertheless, the orifice of the distal shunt catheter, most often located in the peritoneal cavity, is susceptible to blockage as previously described. This may be due to mechanical obstruction by omental fat, proteinaceous debris, intra-abdominal fluid, or intra-abdominal adhesions. The first does not, by any means, infer that a fat plug is physically lodged into the orifice, but that the catheter orifice is merely abutting a mass of omental fat with resultant increase in outflow resistance. This may be the physical equivalent of a local increase in intra-abdominal pressure with resultant increased TOR. Similarly, the catheter orifice may be situated between loops of bowel or between bowel and the abdominal wall itself with a local increase in outflow resistance, independent of measured intra-abdominal pressure (normally 0 cm H2O or slightly negative). Also, fluid pockets within the peritoneal cavity may at their depths, possess elevated hydrostatic pressures that too could increase outflow resistance of a resident catheter. It must be understood that only a mild increase in local outflow resistance may result in a malfunction given the fact that intracranial pressure in adult hydrocephalics (with normal pressure hydrocephalus) is only 8-10 cm H2O. This is manifest in the fact that there is an increased incidence of distal shunt malfunction in constipated patients. This is exclusive of those local factors detailed above and is purely a function of elevated (global) intra-abdominal pressure.

A variety of devices for securing catheters and/or preventing catheter malfunction are described in the prior art.

U.S. Pat. No. 5,584,314, issued Dec. 17, 1996, to Dan Bron, describes a self-cleaning inlet head for a fluid. This patent describes an in-line device for the ventricular (proximal) catheter that, by combining mechanical and hydraulic action, effectively loosens and sweeps away debris within the catheter lumen. The mechanism is based upon a piston which slides within the catheter lumen in direct contact with potentially debris-laden cerebrospinal fluid. Though elaborate in design, such a mechanism, by virtue of its intricate structure and reliance on small mobile components, may be susceptible to mechanical failure. And as the device is placed in line with the ventricular catheter, it is most appropriately utilized in the pediatric population as the majority of shunt malfunctions in this population are due to proximal catheter obstruction. Bron does not address the etiology of shunt malfunction in adults, namely occlusion of the distal catheter which typically is inserted into the peritoneal cavity (abdominal cavity).

U.S. Pat. No. 4,382,445, issued May 10, 1983, to Michael Sommers, describes an improved end plug for use in various shunt systems. In the slit valve, having a plurality of elongated slits positioned at the distal end of the shunt system, the end plug is provided with a central section filling the distal end up to a position adjacent to the distal edge of the slits of the valve. Such a plug does not interfere with the normal operation or pressure sensitivity of the valve, but rather acts as a means to decrease shunt malfunctions due to distal end plugging by proteinaceous build-up and other sedimentation and debris in the dead spot between the slit and the plug. While it may be the case that said plug reduces the incidence of malfunction within those shunts incorporating slit valves, such valves in general are associated with a higher incidence of distal malfunction. According to Cozzens, et al (J Neurosurg. 1997 November; 87(5):682-6), the peritoneal end of the shunt system was obstructed in 31 of 140 shunt malfunctions analyzed. In every case in which the peritoneal end was obstructed, some form of distal slit was found: either a distal slit valve in an otherwise closed catheter or slits in the side of an open catheter. No instances were found of distal peritoneal catheter obstruction when the peritoneal catheter was a simple open-ended tube with no accompanying side slits. It is concluded therefore that side slits in the distal peritoneal catheters of VP shunts are associated with a greater incidence of distal shunt obstruction. Such valves are less often utilized as a result.

U.S. Pat. No. 5,405,316, issued Apr. 11, 1995, to Gary Magram, describes a cerebrospinal fluid shunt including an inner tube for supplying the fluid only from brain ventricles to the peritoneum region of a subject and an outer tube arranged so the fluid remains resident in the outer tube without flowing to the peritoneum region. Fluid in the outer tube exerts pressure through a wall of the inner tube on the fluid in the inner tube to regulate flow of the fluid through the inner tube to the peritoneum region.

Some devices serve to anchor catheters in a fixed location and at certain incident angles.

U.S. Pat. No. 6,562,005, issued May 13, 2003, to David Donath, describes a catheter button system containing primary and secondary apertures.

U.S. Pat. No. 6,554,802, issued Apr. 29, 2003, to Robert Pearson, Douglas O. Hankner, and Weiping Yu, describes an anchor for securely positioning a catheter intended to deliver drug or other medicaments to a desired position in tissue wherever found in the body or in epidural or intrathecal space of a spinal cord or brain. The body of said device has a slot that extends through the body between the wings. The wings interact with the slot to radially compress a traversing catheter within the lumen of the tubular body, holding the catheter in a fixed position. In addition, the wings allow the device to be sutured to tissue to secure and fix the device to tissue.

U.S. Pat. No. 7,014,627, issued Mar. 21, 2006, to Steven Bierman, describes a device for securing a medical device to a patient, specifically to a patient's skin.

U.S. Pat. No. 5,451,212, issued Sep. 19, 1995, to Erik Anderson, relates generally to a device for securing a tube, such as a medical catheter or feeding tube, at the site of a body opening. More specifically, the invention is directed to a bumper retention device for retaining a feeding tube in an angular fixation externally against the skin of a patient, so as to prevent slippage, dislodgement, or unnecessary migration of the feeding tube into the stomach, small intestine or other internal body cavity of a patient. The bumper retention device may be used with a conventional catheter or feeding tube and a conventional retention bar. The bumper retention unit comprises a retention stem portion connected to a loop portion. The loop portion is placed around the outer diameter of the feeding tube and when the tube is bent, the stem portion is inserted into an end aperture of the retention bar so that the tube is retained at an approximately 90 degree angle.

U.S. Pat. No. 5,722,959, issued Mar. 3, 1998, to Steven F. Bierman, describes a securement device which retains an indwelling catheter and allows for easy adjustment of the catheter incident angle while securely fixing the catheter at a desired incident angle once set by a health care provider.

U.S. Pat. No. 6,770,055, issued Aug. 3, 2004, to Steven F. Bierman, Wayne T. Mitchell, and Richard A. Pluth, provides a method using a simply-structured anchoring system that secures a catheter in a fixed position, but easily releases the catheter for dressing changes or other servicing. The anchoring device comprises an anchor and a retainer. The retainer is attached to an upper surface of the anchor and comprises a base, a cover and a post. The base is disposed on the upper surface of the anchor and the cover is connected to the base so as to move between an open and a closed position. When the cover is in the closed position, it lies above at least part of the base. The post is attached movably to either the base or the cover and is arranged so as to lie at least partially between the cover and the base when the cover is in the closed position. When securing a medical article, the cover is placed in the open position and the medical article placed onto the retainer. The cover may then be closed over the medical article and the anchoring device attached to the patient.

U.S. Pat. No. 7,056,286, issued Jun. 6, 2006, to Adrian Ravenscroft and Stephen J. Kleshinski, provides a method and apparatus for anchoring a medical implant device after the device has been brought to rest at a desired position within a blood vessel or other body passageway. An anchor delivery system is provided which houses one or more uniquely configured expandable anchors which are connected to the medical implant device. The anchors remain housed in a non-expanded configuration until after the medical implant device has come to rest in a desired position within the body, and then the anchors are positively propelled through a body wall from a first side to a second side where each anchor expands outwardly on opposite sides of an anchor shaft. To positively propel the anchors, a drive shaft for the anchor shafts extends back to a triggering unit which, when activated, causes the drive shaft to drive the anchor shafts in a direction which results in propulsion of the anchors through the body wall.

U.S. Pat. No. 7,090,660, issued Aug. 15, 2006, to Jerry H. Roberts and Zane D. Myers, provides a patient medical tubing and catheter anchor and support for permanently, securely anchoring and supporting medical tubing to a patient's body including a longitudinally-extending conformable base having a non-adhesive upper surface and a lower surface including a medical grade adhesive thereon. An attachment member is connected to the upper surface of the base to provide a supporting surface for receiving and supporting the tubing. The attachment member includes a center portion and at least one longitudinally-extending locking strip extending from one end of the center portion and at least one other longitudinally-extending locking strip extending from the other end of the center portion. The locking strips are foldable over the center portion to encapsulate a portion of the tubing between the upper surface of the locking strips and the upper surface of the center portion.

U.S. Pat. No. 7,270,650, issued Sep. 18, 2007, to Mary M. Morris, Duane Gerald Frion, Jeff Novotny, Douglas Hankner, and Stuart Lahtinen, provides a catheter system and method for intracranial infusion of therapeutic substances to a patient. An anchor formed of generally flexible, elastomeric material is used to mount the catheter to the outer surface of the skull of the patient. The anchor has a through hole for receiving the catheter, a channel, extending substantially from the through hole, into which a portion of the catheter may be inserted to retain the portion substantially parallel to the surface of the skull, and at least one flange for engaging the outer surface of the skull. An anchor clip may be provided to engage the anchor adjacent the through hole to further stabilize the catheter.

Currently, no implantable device exists to maintain CSF flow in the distal limb of a shunt catheter. The distal limb of the shunt system may be implanted laparoscopically into the abdominal cavity in order to strategically locate the distal catheter orifice in a region free of potentially obstructive fat, fluid pockets, proteinaceous debris, or intra-abdominal adhesions (from prior surgery). Such a technique has been described previously (Li et al. Minimally Invasive Therapy 16(6):367-368 2007). However, the catheter tends to migrate as a result of peristaltic movement (of the bowel) and bodily position changes. This potentially mitigates the advantage of laparoscopic implantation as the catheter may migrate into an unfavorable position with potential for obstruction.

The prior art does not address the root problem of catheter migration. Although the prior art describes numerous devices to affix catheters, there remains a need for a discrete, implanatable device used to affix CSF shunt catheters in an intra-abdominal location free of potentially obstructive tissue or debris. By doing so, the device of the present invention maintains the patency of the distal catheter lumen and reduces the incidence of shunt malfunction.

### SUMMARY OF THE INVENTION

In accordance with the present invention, there is provided an implantable system and device that functions to maintain cerebrospinal fluid (CSF) shunt patency. Although the preferred embodiment relates to CSF shunt catheters, it is contemplated that the system and device of the invention would be useful in other types of shunt catheter systems as well.

The implantable system can have three components. In a preferred embodiment, the system is composed of a shunt catheter, a circumferential cuff through which the shunt catheter traverses, and flanges. The implantable device can have two components. In another preferred embodiment, the device is composed of a circumferential cuff through which the shunt catheter traverses and flanges. The cuff may be composed of nylon mesh (commonly used material in general surgical procedures) or alternate materials. The inner diameter of the cuff approximates the outer diameter of the CSF shunt distal catheter. This will guarantee a snug, secure fit between the cuff and the shunt catheter while concomittantly preserving CSF flow within the catheter lumen. The shunt catheter should remain in a fixed position within the cuff until physiologic scar formation occurs, further securing the device and traversing catheter, as a unit or system, to the target tissue or anatomic structure.

Flanges are affixed to the circumferential cuff. The flanges and cuff are constructed of like material in one embodiment and may be positioned at right angles to the bore axis of the cuff. Additionally, the flanges may be positioned at angles, in all planes, relative to the bore axis of the cuff and may vary in shape and size. In an alternate embodiment, the flanges and cuff are constructed of dissimilar materials. Materials such as VELCRO®, TEFLON®, or nylon mesh may be utilized. Alternatively, more rigid materials, such as urethane or silicone, may be utilized. The flanges may be used alone or may accept both sutures and staples. The flanges function to anchor the system to the target tissue or anatomic structure at the selected site. In a preferred embodiment, the target anatomic structure is the falciform ligament. The falciform ligament is a reflection of peritoneum that courses from the anterior abdominal wall to the liver (and in fact divides the liver into right and left lobes) in the sagittal plane.

An alternate embodiment is composed of a circumferential cuff and a disc or button of similar or dissimilar material as the flanges described above. The cuff is set within the disc's center and traverses it at a 90 degree angle to the flat plane of the disc. The disc may be used alone or may accept suture and staples (circumferentially) and allows for fixation of the system to the target tissue or anatomic structure at the selected site. The target anatomic structure is often the falciform ligament. This system may be secured to alternate sites/structures within the peritoneal cavity (i.e. reflections of peritoneum referred to as “ligaments” or the abdominal wall itself).

Further embodiments may be composed of a more elongated cuff with opposing paired flanges, discs or a combination of a disc and a flange. Staple or suture fixation to the target anatomic structure may or may not be used with such embodiments. For example, the flanges on the leading edge of the cuff in a dual-flanged embodiment, composed of deformable material, may be passed through the created fenestration in a falciform ligament. The flanges will serve to resist potential catheter pullout by their expansibility and large size relative to the created fenestration in the falciform ligament. Similarly, those flanges on the trailing edge of the cuff will prevent the distal shunt catheter from advancing into the right abdominal gutter. In this recess, bordered laterally by the abdominal wall and medially by the liver, obstructive debris or fluid pockets may reside and potentially obstruct the distal tip of the shunt catheter. The embodiments may have a directional bias for implantation as can be observed in the figures.

The implantable systems and devices of the invention serve to secure the distal limb of the shunt catheter to a site that is relatively free of potentially obstructive soft tissue, fat, proteinaceous debris, intra-abdominal fluid pockets and intra-abdominal adhesions. The distal catheter tip is therefore less susceptible to blockage and the incidence of shunt malfunction is transitively reduced. In a preferred embodiment of the described method, the distal shunt catheter is passed through a fenestration created in the falciform ligament. A short length of distal shunt catheter emanates through the created fenestration in the ligament and ultimately lays on the superior serosal surface of the right lobe of the liver. This is a site relatively free of potentially obstructive soft tissue, fat, proteinaceous debris, free fluid and intra-abdominal adhesions in which the catheter may lay. In addition, the distal catheter tip may be better exposed to the negative pressure of the subdiaphragmatic space, with resultant enhancement of CSF flow through the shunt system as CSF shunts are typically pressure-driven systems in which the difference between intracranial and intra-abdominal pressure drives CSF flow.

In addition, the system and/or device may be affixed to the peritoneal limb of lumboperitoneal shunts used to treat pseudotumor cerebri (clinical evidence of elevated intracranial pressure in the context of normal cerebral anatomy, as demonstrated by neuroradiographic evaluation). Lumboperitoneal shunts are typically inserted into the lumbar spine and shunt fluid into the peritoneal cavity. Similar to ventriculoperitoneal shunts, they are subject to malfunction.

There is also a need for a method to implant the systems and devices of the invention, in particular, a minimally-invasive method that does not promote formation of scars and intra-abdominal adhesions, which are potential etiologies of shunt catheter malfunction. Generally, the described system and/or device is implanted laparoscopically during primary shunt placement or revision procedures. Other methods of implantation may be utilized as known to those skilled in the art.

Laparoscopic implantation of the distal limb of the CSF shunt catheter system may reduce the incidence of shunt malfunction by optimizing catheter placement in a region free of potentially obstructive tissue or debris. However, the conferred benefit is likely temporary. Peristaltic motion and bodily movement promote catheter migration into less favorable positions within the abdominal cavity. The catheter may become sequestered within loops of bowel and mesenteric fat or within a fluid pocket with locally elevated hydrostatic pressure. By either, CSF outflow resistance is increased resulting in shunt malfunction. It is much less often the case that the distal catheter tip is physically plugged with proteinaceous fat or debris. In fact, this is rarely observed intra-operatively during distal revision surgical procedures. During the vast majority of such procedures, the surgeon removes the distal catheter from the abdominal cavity only to find a functioning shunt system evidenced by CSF fluid egress from the catheter tip. Several centimeters of distal catheter are cut from the tip and the catheter is again placed into the abdominal cavity. Whether this is performed laparoscopically or another technique is irrelevant, because the root problem of catheter migration has not been addressed.

The implantable systems and methods of the invention solve this problem by providing fixation of the distal shunt catheter to an anatomic structure, which suspends it in a site free of potentially obstructive soft tissue, fat, proteinaceous debris, intra-abdominal fluid pockets and intra-abdominal adhesions.

From the description a number of advantages of the invention become evident:

(a) by reducing the incidence of shunt malfunctions, the systems and devices of the invention reduce the number of surgical revision procedures necessary, translating into a reduction of surgical morbidity and cost;
(b) the systems and devices of the invention are easily implanted either at the time of initial surgery or during a revision procedure;
(c) the system and devices are preferably implanted laparoscopically. This method minimizes intra-abdominal adhesion formation associated with open abdominal shunt catheter surgery;
(d) as the devices are externally coupled to the distal shunt catheter, there are no intra-luminal components capable of causing a shunt malfunction; and
(e) the system and devices are not subject to malfunction as caused by accumulation of proteinaceous debris and/or fat.

Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a side view of the implantable device with a traversing peritoneal shunt catheter (cut ends);

FIG. 2 is an oblique view of the implantable device with a traversing peritoneal shunt catheter (cut ends);

FIG. 3 is a front view of an alternate embodiment of the implantable device with a traversing peritoneal shunt catheter (cut ends);

FIG. 4 is a side view of an alternate embodiment of the implantable device with a traversing peritoneal shunt catheter (cut ends);

FIG. 5 is a front schematic view of a patient showing the implantable system in final position;

FIG. 6 is a side view of a patient illustrating key anatomic structures;

FIG. 7 is a front schematic view of a patient with laparoscopic trochar sites demarcated;

FIG. 8 is an in situ view, from an intraperitoneal location, of the distal limb of the shunt catheter as introduced into the abdominal cavity prior to creation of the fenestration in the falciform ligament;

FIG. 9 is an in situ view (from an intraperitoneal location) of the fenestration technique;

FIG. 10 is an in situ view of the implantable system in place, affixed to the falciform ligament;

FIG. 11 is a side view of an alternate embodiment of the implantable system affixed to the falciform ligament;

FIG. 12 is a top view of an alternate embodiment of the implantable device with traversing distal shunt catheter;

FIG. 13 is a side view of an alternate embodiment of the implantable system with dual, conical flanges;

FIG. 14 is a side view of an alternate embodiment of the implantable system in a hybrid configuration of both flange and disc;

FIG. 15 is an oblique view of an alternate embodiment of the implantable device;

FIG. 16 is a side view of an alternate embodiment of the implantable system in a hybrid configuration of both disc and struts;

FIG. 17 is a side view of an alternate embodiment of the implantable system;

FIG. 18 is a side view of the implantable system in a hybrid configuration of bio-adsorbent flange and disc; and

FIG. 19 is a side view of an alternate embodiment of the implantable system having inflatable flanges.

### DEFINITIONS/ABBREVIATIONS

The following list defines terms, phrases, and abbreviations used throughout the specification. Although the terms, phrases, and abbreviations are listed in the singular tense, this list is intended to encompass all grammatical forms. Preferred embodiments are exemplary only and are not intended to limit the scope of the invention.

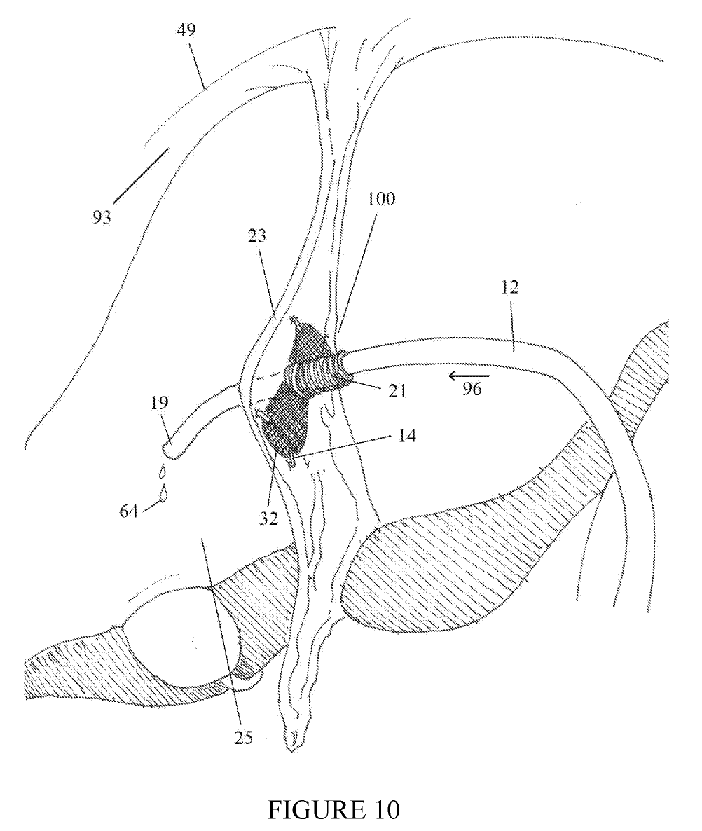

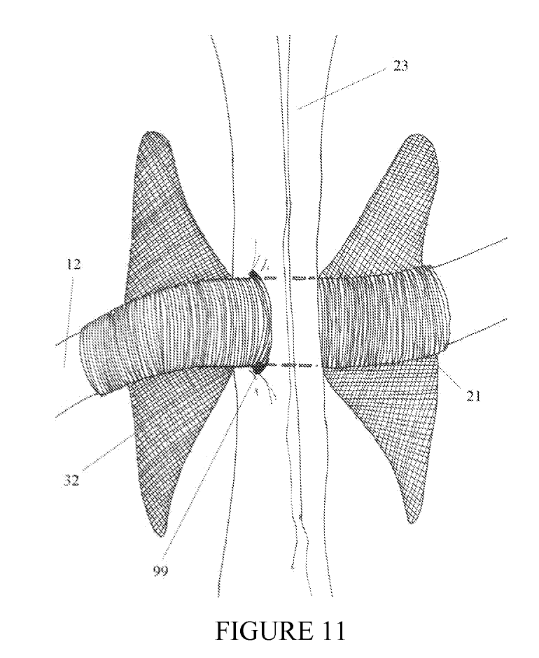

As used herein, the term “implantable system” refers to a system that is implanted internally in the body of a subject for transport of bodily fluids from one location to another within the body. The implantable system includes a shunt catheter and means for securing the shunt catheter to prevent malfunction. The implantable system is illustrated in the figures, in particular, FIG. 10, wherein it is designated number 100.

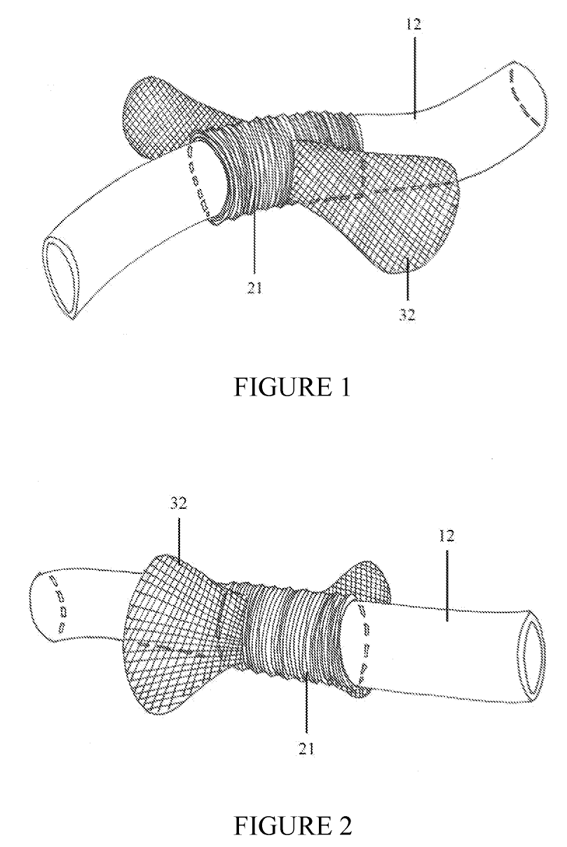

As used herein, the term “implantable device” refers to a device that is implanted internally in the body of a subject to prevent malfunction of a shunt catheter due to obstruction of the catheter opening. The implantable device includes means for holding a limb of a shunt catheter and means for affixing the shunt catheter to an anatomic structure. The implantable device is illustrated in the figures, for example, in FIG. 1, wherein cuff 21 is shown with outwardly expanding flanges 32. The cuff can be integral or separate from the flanges.

As used herein, the term “shunt catheter” refers to a hollow tube employed to drain fluids from body cavities.

As used herein, the term “patency” refers to the condition of not being blocked or obstructed. Thus, a patent shunt catheter is not blocked or obstructed.

As used herein, the phrase “distal shunt catheter (subcutaneous)” refers to the portion of the shunt catheter distal to the valve that is tunneled subcutaneously from the cranial region to the abdominal region. For reference purposes, it is designated number 9.

As used herein, the phrase “distal shunt catheter (intraperitoneal)” refers to the distal portion of the shunt catheter after it has been introduced into the abdominal cavity. For reference purposes, it is designated number 12.

As used herein, the abbreviation “CSF” refers to cerebrospinal fluid. Cerebrospinal fluid is the clear fluid that circulates through the ventricles of the brain, the cavity of the spinal cord, and the subarachnoid space lubricating the tissues and protecting them from injury. For reference purposes, droplets of CSF are designated number 64.

As used herein, the term “subject” or “patient” refers to a human being or animal being treated or capable of being treated with the implantable system and/or devices of the instant invention.

As used herein, the term “hydrocephalus” refers to the pathological accumulation of cerebrospinal fluid in the ventricles of the brain.

As used herein, the term “primary surgery” refers to the initial implantation of a shunt catheter.

As used herein, the term “revision surgery” refers to a surgical procedure secondary to the initial implantation of a shunt catheter that is necessary due to malfunction of the shunt. The methods, systems, and devices of the instant invention can be used either in primary or revision surgery.

As used herein, the term “fenestrate” refers to creating an opening. For example, a fenestrated ligament has an opening to accommodate a limb of a shunt catheter.

As used herein, the term “laparoscopic procedure” refers to an examination or surgery performed with a laparoscope. A laparoscope is a slender, tube-shaped endoscope that is inserted through a small incision in the abdominal wall. The systems and devices of the instant invention can be implanted using a laparoscopic procedure.

As used herein, the term “trocar” refers to a commonly available sheath, inserted percutaneously into the peritoneal cavity, through which surgical instruments are passed.

As used herein, the term “anatomic structure” refers to abdominal tissue to which the shunt catheter can be affixed. An anatomic structure with limited function is preferred.

As used herein, the term “ligament” refers to a sheet or band of tough, fibrous tissue that connects bones and/or cartilages or supports muscles and/or organs.

As used herein, the term “falciform ligament” refers to the ligament that attaches part of the liver to the diaphragm and the abdominal wall and divides the liver into left and right lobes. The falciform ligament is the preferred anatomic structure to which the systems and devices of the invention are affixed. For reference purposes, the falciform ligament is designated number 23.

As used herein, the term “hepatogastric ligament” refers to the ligament that attaches the liver to the lesser curvature of the stomach.

As used herein, the phrase “relatively free of potentially obstructive tissue, proteinaceous debris, and fluid” describes the site at which the systems and devices of the invention are secured; i.e. the site is open, with very little or no tissue, debris, and/or fluid present to obstruct the flow of fluid from the shunt catheter.

As used herein, the term “cuff” refers to a hollow tube through which a shunt catheter can traverse. The cuff has an inner diameter that approximates the outer diameter of a shunt catheter such that when the cuff encircles and holds the shunt catheter a secure fit is obtained while concomittantly not impairing liquid flow within the catheter lumen. For reference purposes, the cuff is designated number 21.

As used herein, the term “disc” refers to a circular material through which a cuff can traverse. In a preferred embodiment, a cuff traverses the disc at a 90° angle to the flat plane of the disc. The disc is also referred to as a button. The terms “disc” and “button” are used interchangeably herein. For reference purposes, the disc is designated number 42.

As used herein, the term “flange” refers to a projection used to strengthen an object, hold it in place, or provide a base for attachment to another object. In the instant case, a flange projects from a circumferential cuff and functions to hold a catheter in place and/or accept staples or sutures for attachment of a catheter to an anatomic structure. For reference purposes, the flange is designated number 32.

As used herein, the term “struts” refers to structural elements used to brace or strengthen by resisting compression. In the instant case, the struts brace a catheter and prevent disengagement of the catheter from its fixed position. For reference purposes, the struts are designated number 104.

As used herein, the term “kit” refers to a collection of supplies for a specific purpose, i.e. supplies to construct and implant the systems and devices of the invention. The kit of the invention may be provided in a closed package with written instructions for use.

### DETAILED DESCRIPTION OF THE INVENTION

For purposes of clarity and brevity, like elements and components bear the same designations and numbering throughout the Figures.

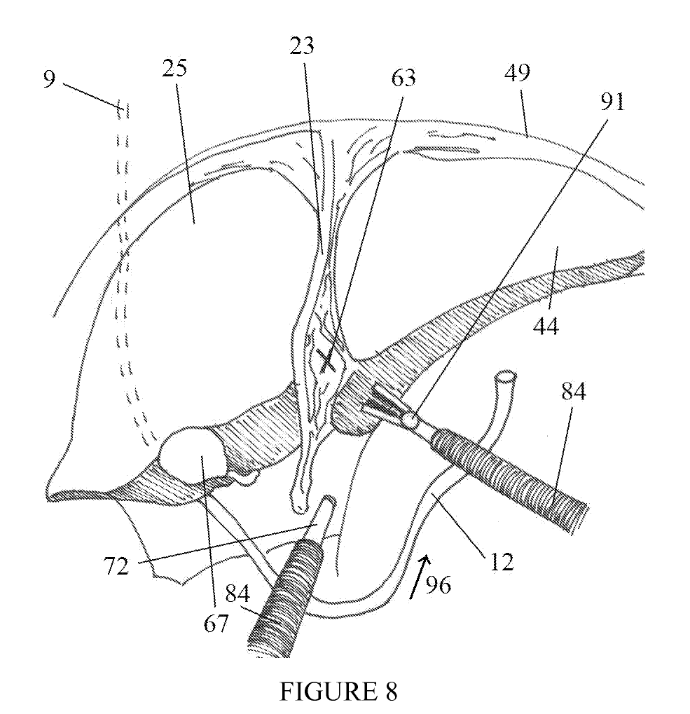

FIG. 1 is a side view of an implantable device with a traversing peritoneal shunt catheter. In a preferred embodiment, cuff 21 is a circumferential sleeve of pliable material such as VELCRO®, TEFLON® or nylon mesh. Alternate materials such as urethane or silicone can also be utilized. Radio-opaque materials or dyes can be incorporated into cuff 21 to allow for x-ray localization of the implantable system. Generally, cuff 21 has a circular, cross-sectional shape. Distal shunt catheter (intraperitoneal) 12 traverses circumferential cuff 21. The inner diameter of cuff 21 approximates the outer diameter of distal shunt catheter (intraperitoneal) 12 thereby assuring a tight interface between said elements. Such a snug, secure fit is necessary to prevent catheter dislodgement and resultant catheter migration. To promote this tight interface, distal shunt catheter (intraperitoneal) 12 should interface with cuff 21 throughout the entire length of said cuff. In a preferred embodiment, the length of cuff 21 is 1 cm. Alternate lengths may be utilized as long as cuff 21 receives a sufficient length of distal shunt catheter (intraperitoneal) 12 to inhibit movement of said catheter. In a preferred embodiment, a pair of flanges extend from the outer surface of cuff 21. Alternate configurations may be utilized (i.e. single or more than two flanges 32, different spatial arrangement of flanges). Each flange 32 is composed of a material such as VELCRO®, TEFLON® or nylon mesh. Alternate materials may be utilized such as urethane or silicone. Flange 32 extends from the center of the outer surface of circumferential cuff 21 at a 90° angle. The flanges assume a wing-like configuration, oriented vertically and perpendicular to the longitudinal plane of cuff 21. They may be formed separately from cuff 21 and then coupled to same. In a preferred embodiment, as illustrated in FIG. 10, flange 32 can accept either a staple or suture 14 and serves to anchor said device to an anatomical structure such as falciform ligament 23. Flanges can also be utilized without staples or sutures. The implantable systems and devices restrict, and may completely prevent longitudinal, rotational and transverse movement of the retained section of distal shunt catheter (intraperitoneal) 12. In a preferred embodiment, flange 32 is composed of easily deformed, pliable material, potentially in a mesh configuration. This maximizes the interface between said flange 32 and falciform ligament 23, as flange 32 will conform to the surface of falciform ligament 23. Secondarily, this will facilitate staple or suture 14 fixation of proposed device to the falciform ligament 23. Additionally, as illustrated in FIG. 8, the pliable material of said flange 32 will allow for easy introduction of the device into the peritoneal cavity via the laparoscopic trochar 84 or an alternate conduit (i.e., “peel-away” catheter).

FIG. 2 is an oblique view of an implantable device with a traversing peritoneal shunt catheter. Cuff 21 does not have a directional bias for receipt of traversing shunt catheter (i.e., it is nondirectional). Typically, 3-6 cm of catheter emanates from the distal end of the device. This may be varied according to the operating surgeon's preference as dictated by patient anatomy (i.e., liver size). The distal shunt catheter is pulled carefully through cuff 21 (to the desired length) prior to the final introduction of the system (device with the shunt catheter) into the peritoneal cavity. It is necessary to “pull” the catheter through cuff 21 as the inner diameter of cuff 21 closely approximates the outer diameter of distal shunt catheter (intraperitoneal) 12. A variety of known techniques may be utilized. Commonly, a suture is tied to the distal catheter tip 19 (FIG. 10) and said suture is passed through cuff 21 of the device. The suture is then used to “pull” distal shunt catheter (intraperitoneal) 12 through cuff 21 until the desired catheter length is reached. Distal shunt catheter (intraperitoneal) 12 is then released from the suture and the system is introduced into the peritoneal cavity as per the method described below.

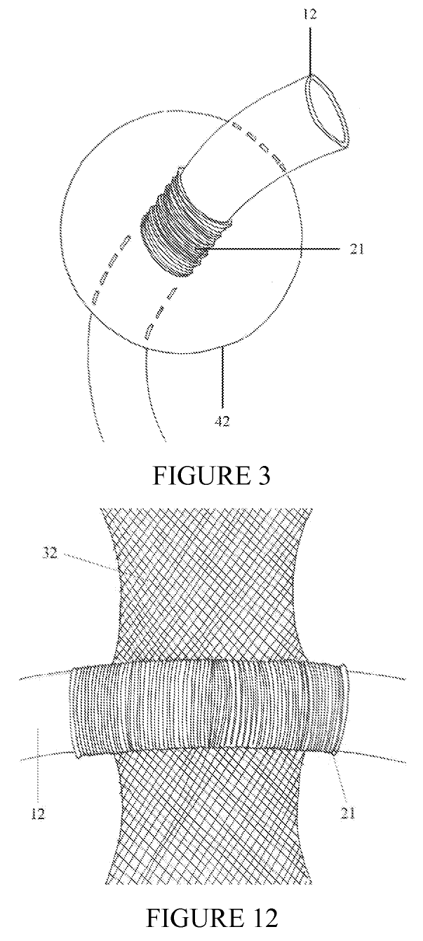

FIG. 3 is a front view of an alternate embodiment of the implantable device with a traversing peritoneal shunt catheter. Anatomy may dictate utilization of the described alternate embodiment. Cuff 21 bisects a round disc (button 42) of similar or disimilar material. VELCRO®, TEFLON® or nylon mesh may be utilized for both cuff 21 and button 42. Alternate materials, such as silicone or urethane, may be utilized as will be clear to those skilled in the art. Cuff 21 bisects said button 42 at a 90° angle to the horizontal plane. Button 42 diameter is typically several times that of the outer diameter of cuff 21. This provides adequate surface area for contact between button 42 and target tissue (i.e., falciform ligament 23) and also provides a large surface for the acceptance of either a staple or suture 14. Disc/button 42 can also be used without staples or sutures.

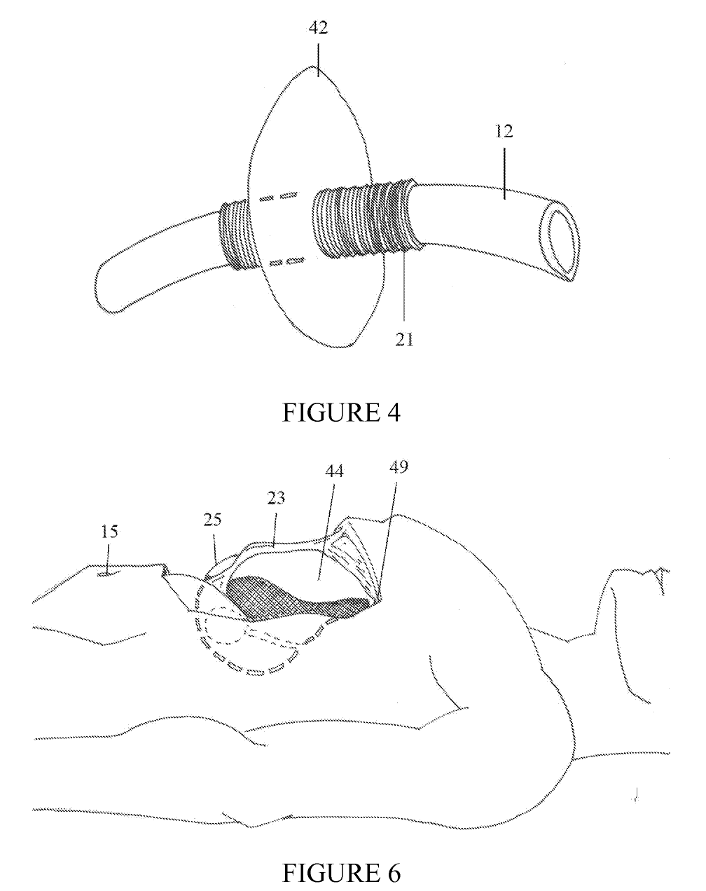

FIG. 4 is a side view of an alternate embodiment of the implantable device with a traversing peritoneal shunt catheter. Distal shunt catheter (intraperitoneal) 12 is introduced into cuff 21 as described previously. In a preferred embodiment, button 42, in a manner similar to flange 32, is composed of an easily deformed, pliable material, potentially in a mesh configuration. This will facilitate introduction of the system (device and traversing catheter) into the peritoneal cavity via the laparoscopic trochar 84 (FIG. 8). As realized from the figure, the disc must be compressed or folded onto itself, minimizing its size prior to its passage through the laparoscopic trochar 84 (preferably, but not limited to, 5 mm in diameter).

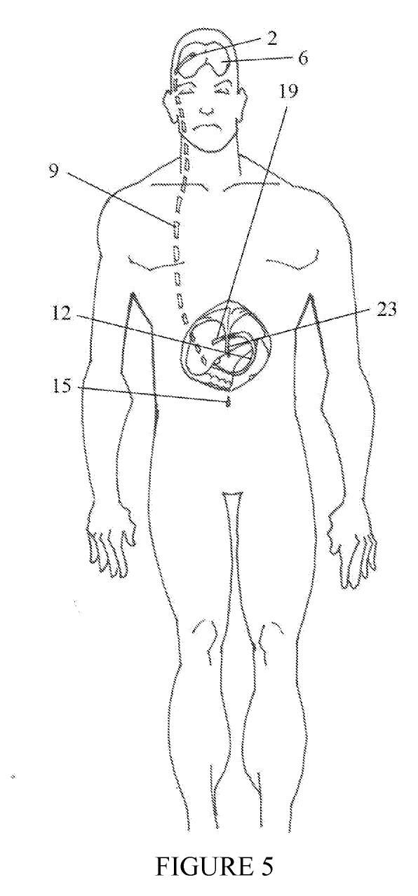

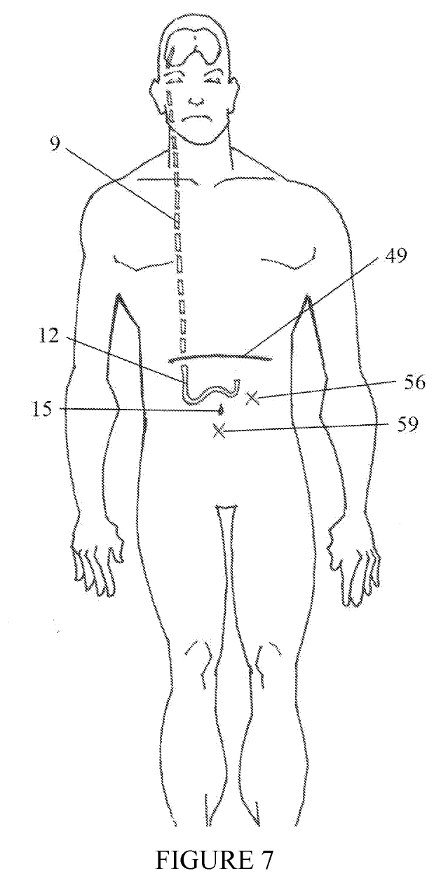

FIG. 5 is a front schematic view of a patient showing the implantable system in final position. In the case of primary or revision procedures, the proximal catheter (ventricular) 2 of the system is placed in lateral ventricle (brain fluid cavity) 6 per standard technique and protocol. Distal shunt catheter (subcutaneous) 9 is then tunneled in the subcutaneous layer from the cranial operative site to the right upper quadrant where it is then introduced into the peritoneal cavity. Alternate sites may be utilized in the case of previous right upper quadrant surgery (and prohibitive scarring). This is performed with a Seldinger (wire-guided) technique and a “peel-away” catheter as has been previously described; for example, Christoforidis et al. American Journal of Neuroradiology 27:1738-1740 2006. The device is then affixed to distal shunt catheter (intraperitoneal) 12 which is subsequently fed through falciform ligament 23 to ultimately lay on the superior surface of the right lobe of the liver 25 (FIG. 6).

FIG. 6 is a side view of a patient illustrating key anatomic structures. Falciform ligament 23 extends from the anterior abdominal wall (its lowest attachment being umbilicus 15) to the superior surface of the liver. It divides the liver into left and right lobes. In a preferred embodiment, falciform ligament 23 is fenestrated. Through the fenestration, a length of distal shunt catheter (intraperitoneal) 12 is fed. Typically 3-6 cm of catheter lays on the superior surface of the right lobe of the liver 25 in subdiaphragmatic space 93 (FIG. 10). Theoretically, such placement better exposes distal catheter tip 19 (FIG. 10) to negative intra-abdominal pressure (during exhalation). This may enhance CSF flow through the system and is an additional advantage of the invention.

FIG. 7 is a front schematic view of a patient with laparoscopic trochar 84 sites demarcated. “Trochars” are commonly available sheaths, inserted percutaneously into the peritoneal cavity, through which surgical instruments are passed. Generally, two 5 mm laparoscopic trochars are utilized. Alternate sized trochars may be used as determined by the operating surgeon. One is placed in the periumbilical region 59 and a second in the left upper quadrant 56. These sites too may be varied according to the surgeon's preference. In a preferred embodiment, 5 mm incisions are made at sites 56 and 59 to accommodate the laparoscopic trochars. In general, site 59 is a camera port and site 56 is an operating port. Strategic placement of the laparoscopic trochars in this manner will facilitate fenestration of falciform ligament 23.

FIG. 8 is an in situ view, from an intraperitoneal location, of the distal limb of the shunt catheter as introduced into the abdominal cavity prior to creation of the fenestration in the falciform ligament. In the case of primary shunt implantation, distal shunt catheter (subcutaneous) 9 is tunneled from the cranial region and received via a small incision (to accommodate the tunneling device). This incision is typically in the right upper quadrant of the abdomen. Alternate sites may be utilized according to the surgeon's preference. CSF flow is confirmed at the distal catheter tip 19 (extracorporeal) by direct observation; CSF flow is illustrated in FIG. 10. Direction of cerebrospinal fluid flow 96 is demarcated. The patent distal catheter limb is subsequently implanted into the abdominal cavity. An exploratory laparoscopy is performed by the operating surgeon utilizing standard technique and protocol. Laparoscopic trochar 84 sites are as above. In a preferred embodiment of this method, the operating surgeon first seeks optimal visualization of the falciform ligament 23. A target site for fenestration in falciform ligament 63 is identified. Generally, this is a point 1-2 cm above the superior serosal surface of the left lobe of the liver 44 at the ligament's midpoint in the sagittal plane. Other points may be selected based on patient anatomy and the surgeon's preference. An avascular site is preferred to minimize potential bleeding during the fenestration procedure. In addition, any intra-abdominal adhesions (i.e. from gallbladder 67 surgery) are lysed or excised at this time in order to minimize any potentially obstructive soft tissue. Next, the distal catheter limb is guided into the peritoneal cavity from its extracorporeal location. Through the incision from which distal shunt catheter (subcutaneous) 9 emanated during the tunneling procedure, a “peel-away” catheter sheath is introduced into the peritoneal cavity. This is performed with a Seldinger (wire-guided) technique. Through the sheath, distal shunt catheter (subcutaneous) 9 is introduced into the peritoneal cavity. At this point the catheter is appropriately referred to as “distal shunt catheter (intraperitoneal) 12.” The described catheter introduction procedure is visually monitored with the lighted laparoscopic camera 72 (inserted prior) and confirmation of catheter position within the peritoneal cavity is obtained.

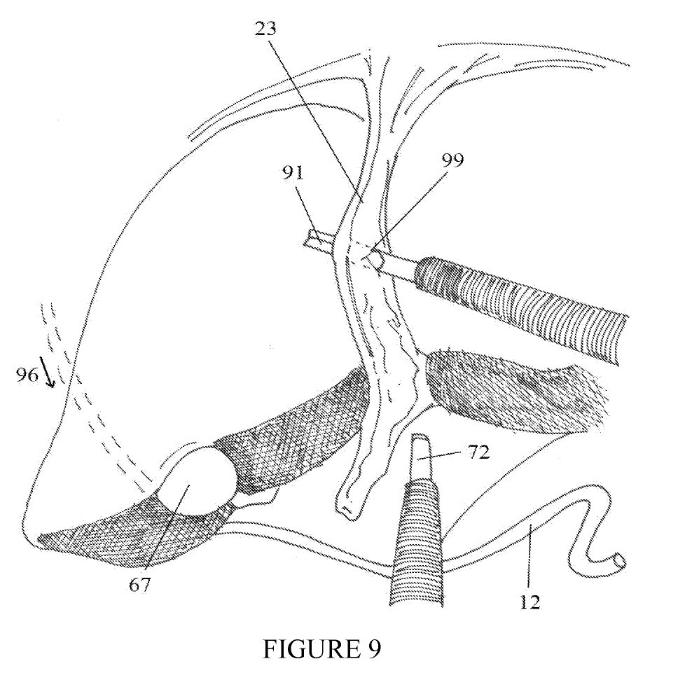

FIG. 9 is an in situ view (from an intraperitoneal location) of the fenestration technique. Falciform ligament 23, identified during the exploratory laparoscopy, is then fenestrated. The operating surgeon inserts a second laparoscopic instrument through a separate abdominal incision/port. Generally, a blunt grasping forceps 91 is inserted and utilized to perform the fenestration in an avascular region of falciform ligament 23. Alternate instruments (i.e., blunt disector) may be utilized depending upon surgeon preference. Blunt instruments generally confer a higher degree of safety. The blunt surface of the closed grasping forceps is carefully pushed through the falciform ligament 23 (typically 1-3 mm in thickness) to create a fenestration. The grasper is then pulled back in a retrograde manner through the newly created fenestration in falciform ligament 99. Typically, the fenestration is performed via a left-to-right pass (of the grasping forceps) through the ligament, from the left upper quadrant to the right. This justifies placement of laparoscopic trochar site: operating port 56 in the left upper quadrant (FIG. 7). A contralateral pass may be utilized if deemed anatomically appropriate. The fenestration is typically created in the midportion of the ligament (sagittal plane), approximately 10-30 mm superior to the liver surface (serosa), 30-60 mm inferior to the dome of abdominal diaphragm 49 (FIGS. 7 and 8). This too may be varied according to surgeon preference. Active ligamentous bleeding about the fenestration, if identified, is addressed with cauterization.

FIG. 10 is an in situ view of the implantable system 100 in place, affixed to the falciform ligament. Distal shunt catheter (intraperitoneal) 12, previously introduced into the peritoneal cavity, is then snared with blunt grasping forceps 91 and pulled through the laparoscopic trochar 84 in a retrograde direction. The catheter will emanate from the trochar (extracorporeally), allowing for its manipulation. Next, the device is applied to the catheter. In any embodiment of the device, the catheter is fed through described cuff 21. 3-6 cm of distal catheter is typically fed through cuff 21. A secure fit is noted between the catheter and cuff 21 prior to final introduction into the peritoneal cavity. Finally, in a preferred embodiment, the flanges of the device are folded over the cuff and as a unit, distal shunt catheter (intraperitoneal) 12 and the device are placed in the jaws of blunt grasping forceps 91. The blunt grasper (with its contents) is then reinserted into the peritoneal cavity via the laparoscopic trochar 84. Distal shunt catheter (intraperitoneal) 12 and the device are visually inspected in preparation for final positioning and securement to falciform ligament 23. Distal shunt catheter (intraperitoneal) 12 is typically passed in a left-to-right direction through the made fenestration in falciform ligament 99 (FIG. 9). This is also preformed with blunt grasping forceps 91 under laparoscopic visualization. Distal catheter tip 19 of distal shunt catheter (intraperitoneal) 12 ultimately lays on the serosal surface of the superior aspect of the right lobe of the liver 25 inferior to the abdominal diaphragm 49. This is a region free of potentially obstructive debris, omental fat, and fluid. Theoretically, cerebrospinal fluid 64 egress there will remain uninterrupted. Generally, 3-6 cm of distal shunt catheter (intraperitoneal) 12 is passed through fenestration in falciform ligament 99. Final position of the distal shunt catheter (intraperitoneal) 12 is visually assessed via the lighted laparoscopic camera 72 (FIG. 9). Ideally, distal shunt catheter (intraperitoneal) 12 should not breach the liver border in any plane or direction.

In one embodiment, the implantable system is affixed to falciform ligament 23. This is illustrated in FIG. 10. In most cases, the device, too large to be passed through the fenestration in falciform ligament 99, abuts the left side of the native falciform ligament 23. Specifically, the flanges (or the body of the disc) contact falciform ligament 23 and serve as anchor points via which the device can be affixed to falciform ligament 23. A laparoscopic stapler (commonly available) is used to staple the flanges (or the body of the disc) to falciform ligament 23. Typically, 2-3 staples are necessary to fixate the device to falciform ligament 23. The integrity of the fixation is inspected by gentle manipulation of the catheter with the blunt grasping forceps 91. In time, the implantable system will be “scarred in” allowing for better fixation of same to the falciform ligament 23.

After a final visual inspection of the system proves satisfactory, the laparoscopic instruments are removed from the peritoneal cavity and the procedure is closed per standard technique and protocol. Typically, there are three abdominal incisions (5 mm wide) requiring closure. However, this may vary depending on the surgeon's preference.

FIGS. 11-19 herein illustrate alternate configurations of the implantable device. The devices utilize variations in flange 32 and disc/button 42 placement, shape, and orientation. Alternate configurations may be utilized to attain securement of distal catheter (intraperitoneal). Patient anatomy (variations of) or surgeon preference may dictate selection of the device best suited for each patient's needs. Implantation of these devices is as illustrated in FIGS. 7-10.

FIG. 11 is a side view of an alternate embodiment of the implantable system affixed to the falciform ligament. Dual flanges in a bow-tie configuration are affixed to central cuff 21. Flange 32 extends at an acute angle from a point several millimeters from the longitudinal center of cuff 21. A gap of several millimeters exists between flange 32 pairs and allows falciform ligament 23 to reside between said flanges (in the sagittal anatomic plane). This device may be introduced into the peritoneal cavity via the laparoscopic method described above. Prior to its introduction, distal catheter (intraperitoneal) is fit with the described device as described previously. Typically, the longitudinal center of the device (gap between flange 32 pairs) is positioned 3-6 cm proximal to the distal catheter tip 19. Blunt grasping forceps 91 is then used to introduce the device into the peritoneal cavity as described above. Distal catheter (intraperitoneal) is then inserted into the created fenestration in falciform ligament 99. The device is then released from the jaws of blunt grasping forceps 91. A single flange 32 pair is located on the distal side of falciform ligament 23 and a second flange 32 pair on the proximal side (over the right and left lobes of the liver respectively). The flanges, previously compressed within the jaws of blunt grasping forceps 91, expand on either side of falciform ligament 23 when the device is released. The flange 32 pair, as a unit, provides resistance to catheter pullout. Falciform ligament 23 is juxtaposed to cuff 21 at the longitudinal center of the device, thereby augmenting resistance to catheter pullout or inadvertant advancement. A surgeon need not use sutures or staples with such an embodiment by virtue of the above “self-expanding” mechanism.

FIG. 12 is a top view of an alternate embodiment of the implantable device with traversing distal shunt catheter. Flange 32 is oriented 90 degrees to cuff 21, parallel to the longitudinal axis of the cuff. This contrasts the orientation of the flanges detailed in FIG. 1. The orientation of flange 32 in FIG. 12 allows for securement of distal shunt catheter (intraperitoneal) 12 to falciform ligament 23 in a side-to-side as opposed to a “transfalciform” configuration. In this case, distal catheter tip 19 may lay on the serosal surface of the left lobe of the liver 44 (as opposed to the right lobe) as it courses back in the direction from whence it came (obviating the need to fenstrate falciform ligament 23). This particular embodiment may also allow for securement of distal shunt catheter (intraperitoneal) 12 to alternate sites within the peritoneal cavity (i.e., ventral abdominal wall). This embodiment is particularly suited for use in patients with unfavorable anatomy (i.e., intra-abdominal adhesions involving the serosal surface of the liver). Flange 32 accepts staple or suture 14.

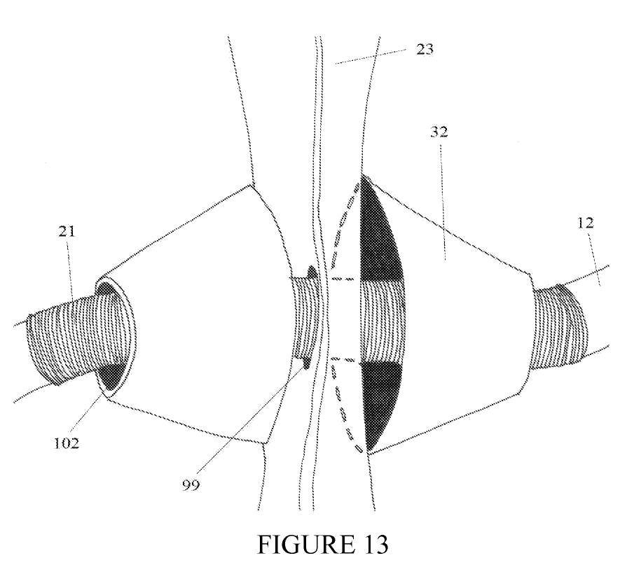

FIG. 13 is a side view of an alternate embodiment of the implantable system with dual, conical flanges. Flange 32 may be composed of silicone or urethane and is shaped conically to form a hollow cone capable of encircling and holding cuff 21. Cuff 21 courses through central axis of flange 32 through orifice 102. In a preferred embodiment, the base of flange 32 is lined with VELCRO®, TEFLON® or nylon mesh to optimize adherence to falciform ligament 23 without requirement for staple or suture 14 (silicone and urethane typically do not scar or integrate into biologic tissue). A second identical flange 32 is similarly bisected by cuff 21 longitudinally. Cuff 21 typically extends 2-3 mm past the conical apex of flange 32. The bases of said flanges are in close approximation to one another (typically positioned 2-3 mm apart). A gap is formed which allows falciform ligament 23 to reside (and integrate itself) between the flanges in the sagittal plane. Implantation of this alternate embodiment is similar to that illustrated in FIG. 11. The conical apex of the leading flange 32, that closest to distal catheter tip 19, is gently pushed through the created fenestration in falciform ligament 99 until the ligament leaflets (coursing in the sagittal plane) are positioned between the bases of the mesh-coated flanges. Upon final positioning of same, the flange 32 pair as a unit provides resistance to distal shunt catheter (intraperitoneal) 12 pullout.

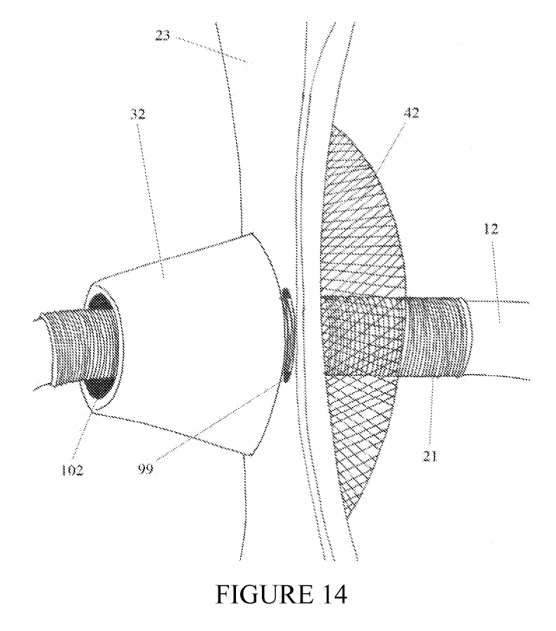

FIG. 14 is a side view of an alternate embodiment of the implantable system in a hybrid configuration of both flange 32 and disc/button 42. In one embodiment, the base of conical flange 32 with adherent mesh is bisected by cuff 21 which similarly bisects disc/button 42. The base of conical flange 32 closely approximates disc/button 42 along the longitudinal axis of cuff 21. The gap between said flange 32 and disc/button 42 (of several millimeters), allows for integration of falciform ligament 23. This embodiment has a directional bias as the diameter of disc/button 42 is of larger diameter than the fenestration in falciform ligament 99. The conical flange 32 is therefore located on the leading edge of the distal shunt catheter (intraperitoneal) 12 and is firstly maneuvered through the created fenestration in falciform ligament 99. Disc/button 42 serves to limit unintended advancement of distal shunt catheter (intraperitoneal) 12 into the right lateral recess of the abdomen. Similarly, conical flange 32 restricts devancement of the distal catheter. Such a configuration may be utilized with or without staple or suture 14. Disc/button 42 may be accepting of either.

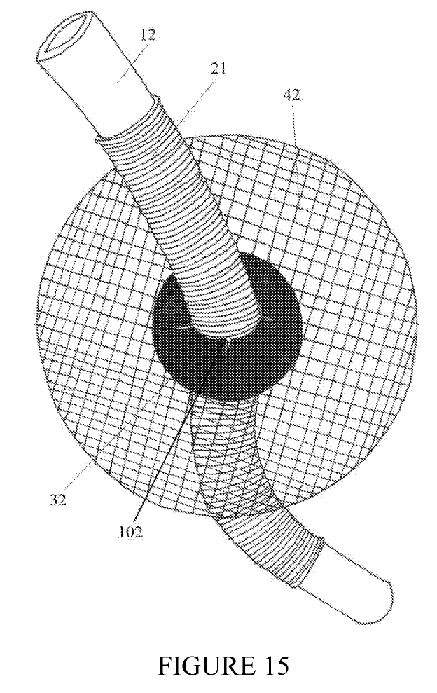

FIG. 15 is an oblique view of an alternate embodiment of the implantable device. Distal shunt catheter (intraperitoneal) 12 courses through cuff 21, itself bisecting conical flange 32 (through orifice 102) and disc/button 42 along a common longitudinal axis. The base of the conical flange 32 (with adherent mesh) is of larger diameter than the created fenestration in falciform ligament 99, as is disc/button 42.

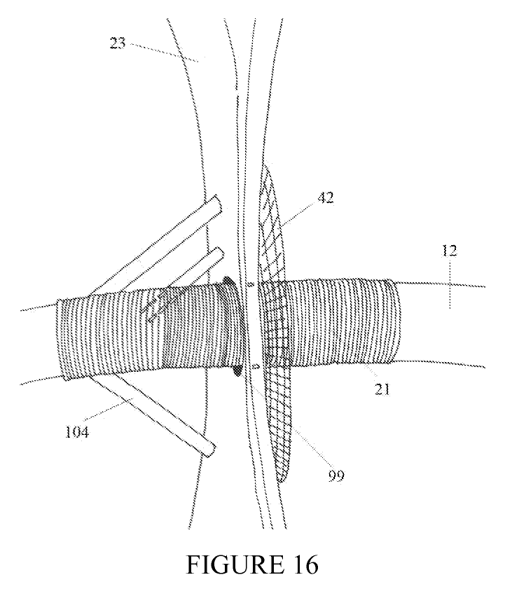

FIG. 16 is a side view of an alternate embodiment of the implantable system in a hybrid configuration of both disc and struts. Flange 32 may be composed of a plurality of urethane or silicone struts 104 affixed to cuff 21 at acute angles. Struts 104 are deformable and may be compressed against cuff 21 during their passage through the created fenestration in falciform ligament 99. Upon their passage (and subsequent release from the jaws of blunt grasping forceps 91), the struts 104, as a group, expand outward from the longitudinal axis of central cuff 21. Collectively, they provide resistance against retrograde pullout of distal shunt catheter (intraperitoneal) 12 through falciform ligament 23. Closely opposed to flange 32 is disc/button 42, also affixed to cuff 21. Flange 32 resists distal catheter advancement in a similar manner as described previously. This alternate embodiment should be positioned midway through the created fenestration in falciform ligament and “capture” the leaves of falciform ligament 23 between flange 32 and disc/button 42. Noteworthy is the directional bias of said embodiment. The orientation of the compressable struts 104 facilitates their passage through the fenestration in falciform ligament 99. Accordingly, this embodiment is applied to distal shunt catheter (intraperitoneal) 12 with flange 32 closest to distal catheter tip 19. Staple or suture 14 may or may not be utilized to affix disc/button 42 to falciform ligament 23 after final positioning.

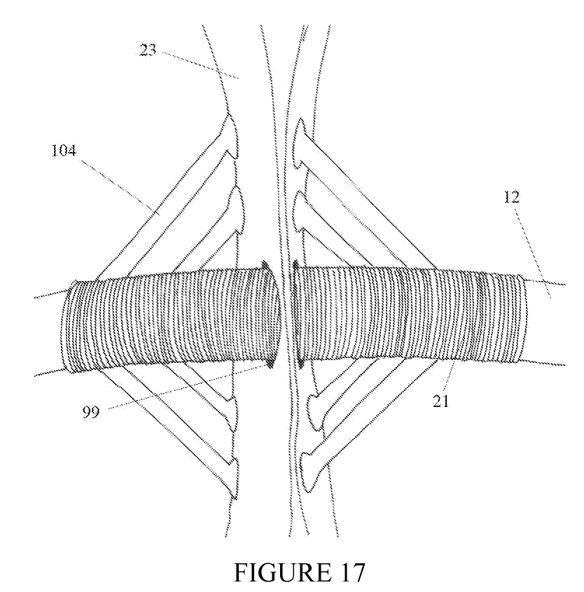

FIG. 17 is a side view of an alternate embodiment of the implantable system. The flanges are struts of similar morphology and construction as those illustrated in FIG. 16 and are affixed to cuff 21 in a mirror configuration. Similarly, the device is positioned into a created fenestration in falciform ligament 99 and “captures” the leaves of falciform ligament 23 between the flange 32 pair. The flanges resist catheter pullout bidirectionally. There is however, no directional bias for application of said embodiment to distal shunt catheter (intraperitoneal) 12 as it symmetrical. Staples or sutures 14 are not utilized with this embodiment.

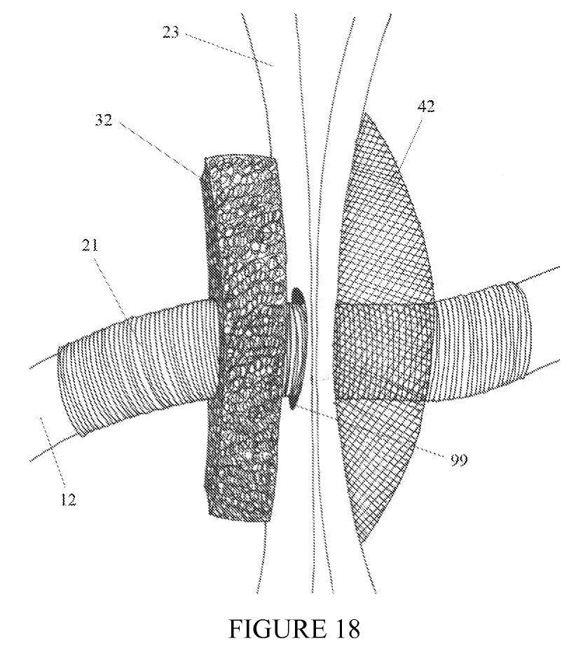

FIG. 18 is a side view of the implantable system in a hybrid configuration of bio-adsorbent flange and disc/button. Flange 32 may be composed of a bio-absorbent material which expands when hydrated (upon implantation into the peritoneal cavity). This “sponge-like” material is affixed to cuff 21 in its dehydrated state. Initially, flange 32 is of significantly lesser volume and is positioned on the distal side of falciform ligament 23 (in most cases, the right) via the created fenestration. Flange 32 is thereafter hydrated, either by physiologic intra-peritoneal fluid or artificially by the surgeon at time of implantation, to promote expansion and subsequent resistance to catheter pullout. Disc/button 42 may or may not accept staples or sutures 14.

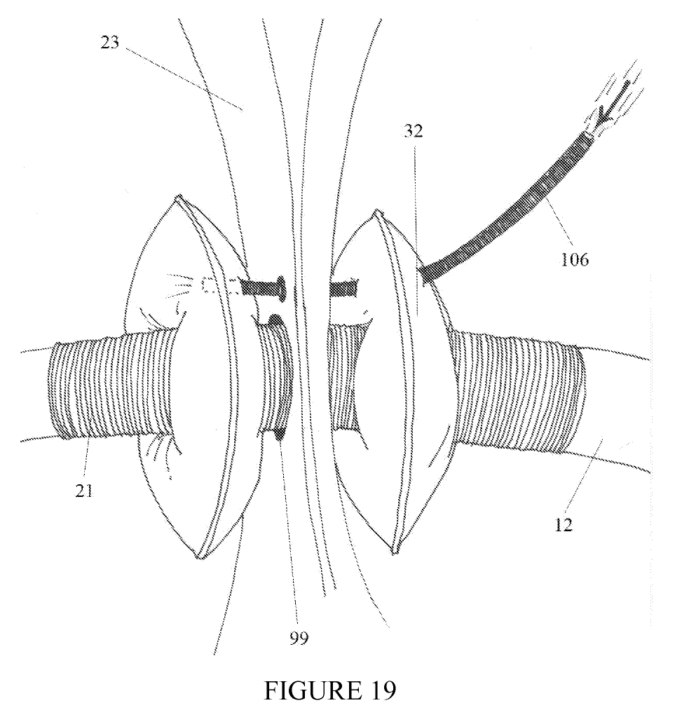

FIG. 19 is a side view of an alternate embodiment of the implantable system having inflatable flanges. Flange 32 may be composed of an inflatable silastic balloon, 1 cm in diameter, affixed to cuff 21. A balloon of like configuration and composition is affixed to cuff 21 several millimeters proximal. The flange 32 pair may be inflated simultaneously via a detachable pneumatic line 106 accessible to the surgeon. The distal flange 32 on cuff 21 is passed through created fenestration in falciform ligament 99 and the flange 32 pair “captures” the leaves of falciform ligament 23. Thereafter, flange 32 (balloon) is inflated and resistance to catheter pullout achieved. The detachable pneumatic line 106, used to inflate the balloons simultaneously, is then removed. The pneumatic line 106 and distal shunt catheter (subcutaneous) 9 are tunneled as a single unit. Both distal shunt catheter (subcutaneous) 9 and pneumatic line 106 emanate from the incision typically made in the right upper quadrant to accommodate the shunt tunneling device. Cuff 21 is then applied to distal shunt catheter (subcutaneous) 9 and, as a pair, distal shunt catheter (subcutaneous) 9 and pneumatic line 106 are passed into the peritoneal cavity via the aforementioned “peel-away” sheath. The sheath will be of larger caliber than those noted previously to accommodate distal shunt catheter (subcutaneous) 9 with the positioned device and pneumatic line 106. The device is then deployed as per the above method utilizing blunt grasping forceps 91. This alternate embodiment, in contrast to those described previously, requires application on distal catheter (subcutaneous) prior to its introduction into the peritoneal cavity. Reiterated, distal shunt catheter (intraperitoneal) 12 is not removed (via laparoscopic trochar 84) from the peritoneal cavity in order to apply the device.

Any of the above-described flange 32 or disc/button 42 configurations may be incorporated into distal shunt catheters in a unitized embodiment as may be recognized by those skilled in the art. This will potentially eliminate the described procedural steps for fitting of the device to distal shunt catheter (intraperitoneal) 12. The unitized embodiment merely will be tunneled (subcutaneously) to the right upper quadrant, introduced into the peritoneal cavity and subsequently affixed to the falciform ligament 23.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The systems, devices, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific, preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An implantable system for securing a shunt catheter to an anatomic structure comprising a shunt catheter, means for holding a limb of said shunt catheter, and means for affixing said shunt catheter to said anatomic structure.

2. The implantable system according to claim 1, wherein said shunt catheter is a cerebrospinal fluid (CSF) shunt catheter or a lumboperitoneal shunt catheter.

3. The implantable system according to claim 1, wherein said anatomic structure is located at a site in an abdominal cavity of a subject.

4. The implantable system according to claim 3, wherein said site is relatively free of potentially obstructive tissue, fat, proteinaceous debris, intra-abdominal adhesions, and intra-abdominal fluid pockets.

5. The implantable system according to claim 1, wherein said anatomic structure is a ligament.

6. The implantable system according to claim 5, wherein said ligament is a falciform ligament or a hepatogastric ligament.

7. The implantable system according to claim 1, wherein said means for holding comprises a circumferential cuff through which said limb of said shunt catheter traverses and wherein said means for affixing comprises at least one flange extending from an outer surface of said circumferential cuff or comprises a disc wherein said circumferential cuff traverses said disc at a ninety degree angle to the flat plane of said disc.

8. The implantable system according to claim 7, wherein said circumferential cuff has an inner diameter that approximates the outer diameter of said shunt catheter.

9. The implantable system according to claim 7, wherein said circumferential cuff is constructed from a material selected from the group consisting of nylon mesh, VELCRO®, TEFLON®, silicone, and urethane.

10. The implantable system according to claim 7, wherein said circumferential cuff includes a radio-opaque material or dye.

11. The implantable system according to claim 7, wherein said flange or said disk is affixed to said anatomic structure by staples or suture.

12. The implantable system according to claim 7, wherein said flange or said disk is constructed from a material selected from the group consisting of nylon mesh, VELCRO®, TEFLON®, silicone, and urethane.

13. The implantable system according to claim 7, wherein said circumferential cuff and said flange or said disc are constructed from the same or dissimilar material.

14. The implantable system according to claim 7, wherein said means for affixing comprises both a flange and a disc.

15. The implantable system according to claim 7, wherein said means for affixing comprises at least one flange extending from an outer surface of said cuff at a ninety degree angle to the longitudinal axis of said cuff.

16. The implantable system according to claim 15, wherein said anatomic structure is an abdominal wall.

17. The implantable system according to claim 7, wherein said means for affixing is a flange shaped as a hollow cone and wherein said flange is capable of encircling and holding said cuff.

18. The implantable system according to claim 17, wherein said flange is constructed from silicone or urethane.

19. The implantable system according to claim 17, wherein the base of said flange includes a material selected from the group consisting of nylon mesh, VELCRO®, and TEFLON®.

20. The implantable system according to claim 7, wherein said flange includes a plurality of struts affixed to said circumferential cuff at acute angle and wherein said plurality of struts expands outwardly from the longitudinal axis of said circumferential cuff.

21. The implantable system according to claim 20, wherein said struts are constructed of silicone or urethane.

22. The implantable system according to claim 7, wherein said flange is constructed of bio-absorbent material which expands when hydrated.

23. The implantable system according to claim 7, wherein said flange is an inflatable balloon.

24. A kit for an implantable system according to claim 1, said kit comprising a shunt catheter, means for holding a distal limb of said shunt catheter, and means for affixing said shunt catheter to an anatomic structure.

25. An implantable device for securing a shunt catheter to an anatomic structure comprising means for holding a limb of said shunt catheter and means for affixing said shunt catheter to said anatomic structure.

26. The implantable device according to claim 25, wherein said shunt catheter is a cerebrospinal fluid (CSF) shunt catheter or a lumboperitoneal shunt catheter.

27. The implantable device according to claim 25, wherein said anatomic structure is located at a site in an abdominal cavity of a subject.

28. The implantable device according to claim 27, wherein said site is relatively free of potentially obstructive tissue, fat, proteinaceous debris, intra-abdominal adhesions, and intra-abdominal fluid pockets.

29. The implantable device according to claim 25, wherein said anatomic structure is a ligament.

30. The implantable device according to claim 29, wherein said ligament is a falciform ligament or a hepatogastric ligament.

31. The implantable device according to claim 25, wherein said means for holding comprises a circumferential cuff through which said limb of said shunt catheter traverses and wherein said means for affixing comprises at least one flange extending from an outer surface of said circumferential cuff or comprises a disc wherein said circumferential cuff traverses said disc at a ninety degree angle to the flat plane of said disc.

32. The implantable device according to claim 31, wherein said circumferential cuff has an inner diameter that approximates the outer diameter of said shunt catheter.

33. The implantable device according to claim 31, wherein said circumferential cuff is constructed from a material selected from the group consisting of nylon mesh, VELCRO®, TEFLON®, silicone, and urethane.

34. The implantable device according to claim 31, wherein said circumferential cuff includes a radio-opaque material or dye.

35. The implantable device according to claim 31, wherein said flange or said disk is affixed to said anatomic structure by staples or suture.

36. The implantable device according to claim 31, wherein said flange or said disk is constructed from a material selected from the group consisting of nylon mesh, VELCRO®, TEFLON®, silicone, and urethane.

37. The implantable device according to claim 31, wherein said circumferential cuff and said flange or said disc are constructed from the same or dissimilar material.

38. The implantable device according to claim 31, wherein said means for affixing comprises a flange and a disc.

39. The implantable device according to claim 31, wherein said means for affixing comprises at least one flange extending from an outer surface of said cuff at a ninety degree angle to the longitudinal axis of said cuff.

40. The implantable device according to claim 39, wherein said anatomic structure is an abdominal wall.

41. The implantable device according to claim 31, wherein said means for affixing is a flange shaped as a hollow cone and wherein said flange is capable of encircling and holding said cuff.

42. The implantable device according to claim 41, wherein said flange is constructed from silicone or urethane.

43. The implantable system according to claim 41, wherein the base of said flange includes a material selected from the group consisting of nylon mesh, VELCRO®, and TEFLON®.

44. The implantable device according to claim 31, wherein said flange includes a plurality of struts affixed to said circumferential cuff at acute angle and wherein said plurality of struts expands outwardly from the longitudinal axis of said circumferential cuff.

45. The implantable device according to claim 38, wherein said struts are constructed of silicone or urethane.

46. The implantable device according to claim 31, wherein said flange is constructed of bio-absorbent material which expands when hydrated.

47. The implantable device according to claim 31, wherein said flange is an inflatable balloon.

48. A kit for an implantable device according to claim 25, said kit comprising means for holding a limb of said shunt catheter and means for affixing said shunt catheter to an anatomic structure.

49. A method for preventing malfunction of a shunt catheter in a subject by implanting a system according to claim 1, said method comprising:
identifying an anatomic structure for affixing said shunt catheter;
introducing said shunt catheter into an abdominal cavity;
preparing said anatomic structure to accept said shunt catheter;
positioning said shunt catheter for engagement with said anatomic structure; and
securing said shunt catheter to said anatomic structure wherein said shunt catheter is secured at a site in said abdominal cavity that is relatively free of potentially obstructive tissue, fat, proteinaceous debris, intra-abdominal adhesions, and intra-abdominal fluid pockets.

50. The method according to claim 49, wherein said method is performed laparoscopically.

51. The method according to claim 49, wherein said shunt catheter is a cerebrospinal fluid (CSF) shunt catheter and said system is implanted in a primary surgical procedure or in a revision surgical procedure for treatment of hydrocephalus.

52. The method according to claim 49, wherein said shunt catheter is a lumboperitoneal shunt catheter and said system is implanted in a surgical procedure for treatment of pseudotumor cerebri.

53. The method according to claim 49, wherein said anatomic structure is a ligament or an abdominal wall.

54. The method according to claim 53, wherein said ligament is a falciform ligament or a hepatogastric ligament.

55. A method for preventing malfunction of a shunt catheter in a subject by implanting a device according to claim 25, said method comprising:
identifying an anatomic structure for affixing said shunt catheter;
introducing said shunt catheter into an abdominal cavity;
preparing said anatomic structure to accept said shunt catheter;
positioning said shunt catheter for engagement with said anatomic structure; and
securing said shunt catheter to said anatomic structure wherein said shunt catheter is secured at a site in said abdominal cavity that is relatively free of potentially obstructive tissue, fat, proteinaceous debris, intra-abdominal adhesions, and intra-abdominal fluid pockets.

56. The method according to claim 55, wherein said method is performed laparoscopically.

57. The method according to claim 55, wherein said shunt catheter is a cerebrospinal fluid (CSF) shunt catheter and said system is implanted in a primary surgical procedure or in a revision surgical procedure for treatment of hydrocephalus.

58. The method according to claim 55, wherein said shunt catheter is a lumboperitoneal shunt catheter and said system is implanted in a surgical procedure for treatment of pseudotumor cerebri.

59. The method according to claim 55, wherein said anatomic structure is a ligament or an abdominal wall.

60. The method according to claim 59, wherein said ligament is a falciform ligament or a hepatogastric ligament.
